(19) United States
(12) Patent Application Publication
(10) Pub. No.: US 2010/0189663 A1
(43) Pub. Date: Jul. 29, 2010

(54) MOUTH RINSE COMPOSITIONS INCLUDING CHEMICALLY MODIFIED SILICA OR SILICATE MATERIALS FOR SUSTAINED DELIVERY TO TOOTH SURFACES

(76) Inventors: Karl W. Gallis, Perryville, MD (US); John McArthur Swazey, JR., San Diego, CA (US); Cozette Ashly B. Pasay, San Diego, CA (US); Michael S. Darsillo, Landenberg, PA (US); Jason Thomas Zapf, Bel Air, MD (US)

Correspondence Address: J M HUBER CORPORATION, 1000 Parkwood Circle, Ste. 1000, PATENT DEPARTMENT, Atlanta, GA, 30339, US

(21) Appl. No.: 12/648,673

(22) Filed: Dec. 29, 2009

Related U.S. Application Data
(60) Provisional application No. 61/275,689, filed on Jan. 24, 2009.

(51) Int. Cl. A61K 8/25; A61K 8/18

(52) U.S. Cl. 424/49

(57) ABSTRACT

Novel mouth rinse (i.e., mouthwash) compositions that permit delivery of silica or silicate materials to the surface of teeth through common mouth rinsing procedures are provided. Such compositions must exhibit proper suspension of the silica or silicate materials (in particulate form) to prevent settling during storage while simultaneously providing proper mouth rinsing properties. Such silica or silicate materials may themselves exhibit any number of therapeutic or aesthetic benefits as long as such materials are easily transferred through mouth rinsing and exhibit proper affinity for deposit on target teeth upon contact therewith. Such silica or silicate materials should exhibit, in one embodiment, certain ionic charge levels as well as sufficiently small particle sizes to permit effective static attraction and eventual accumulation on target tooth surfaces. Methods of utilizing such mouthwashes for breath freshening and/or cleaning, as well as silica or silicate material tooth accumulation, are encompassed within this invention as well.

### CROSS-REFERENCE TO RELATED APPLICATIONS

The present application claims the benefit of priority to U.S. Provisional Application Ser. No. 61/275,689, filed Jan. 24, 2009, entitled “Mouth Rinse Compositions Including Chemically Modified Silica or Silicate Materials for Sustained Delivery to Tooth Surfaces”, the disclosure of which is hereby incorporated by reference in its entirety.

### FIELD OF THE INVENTION

This invention pertains to mouth rinse (i.e., mouthwash) compositions, and more particularly, to mouth rinse compositions that function to decrease tooth sensitivity.

### BACKGROUND OF THE INVENTION

Silica and/or silicate materials are particularly useful in dentifrice products (such as toothpastes) where they function as abrasives and thickeners. In addition to this functional versatility, silica or silicate materials (particularly amorphous precipitated silica materials or calcium silicate particles) also have the advantage, when compared to other dentifrice abrasives (notably alumina and calcium carbonate) of having a relatively high compatibility with active ingredients like fluoride sources (sodium fluoride, sodium monofluorophosphate, etc.). Additionally, silica or silicate materials have been theorized as potential therapeutic agents for dental purposes in other ways, such as dentin tubule blocking, teeth whitening, and remineralization of depleted teeth surfaces. However, in each of these instances, the delivery of silica or silicate materials has been accomplished through dental pastes and creams of high viscosity. Deposition of silica or silicate materials on teeth through other methods has heretofore been avoided due to various difficulties.

Initially, the fact that silica and silicate materials are particulate in nature creates a general problem with delivering such materials from liquid sources. When in liquid form, particulates tend to settle to the bottom of the entire composition, thus providing a suitably homogeneous aliquot of the particulate-containing liquid for transfer and contact with target teeth becomes difficult. In addition, adhesion of specific particulates to tooth surfaces is extremely difficult to achieve when applied from a liquid source since the liquid maneuvers the particulates during rinsing causing removal of some of the particulates when the liquid is expectorated or swallowed. As such, there has been little investigated within the silica industry as to ways to apply silica or silicate materials to tooth surfaces other than in dry form (such as with tooth powders) or dentifrice form (toothpastes and tooth creams).

Mouthwashes have been utilized for various therapeutic benefits in the past. Most commonly, mouthwashes allow for breath freshening through the utilization of alcohols to kill harmful and/or undesirable bacteria in a person's mouth and active ingredients to treat potential problems associated with foul breath, tooth cavities, and gingivitis and by delivery of flavors and scents to mask odors. Mouthwashes generally exhibit a proper low viscosity to allow for full maneuvering within a person's oral cavity and to facilitate expectoration, rather than swallowing. Mouthwashes include large amounts of water-miscible alcohols and water, along with essential oils (eucalyptol, methylsalicylate, and the like) and other agents, such as hydrogen peroxide and tetrapotassium phosphate, which could be harmful if ingested; as such, mouthwashes are not to be swallowed, but expectorated by a user. Expectorating of mouthwash will involve active removal of as much residue from a person's mouth as possible. With a liquid basis, mouthwashes, even after being maneuvered throughout a person's mouth, will most likely include materials that were not only in the mouthwash initially, but also potentially unwanted residues from the user's teeth when active and forceful expectoration occurs. Mouthwashes may also include organic compounds or salts which dissociate in properly acidified media resulting in a propensity to adhere to tooth surfaces in liquefied form.

U.S. Pat. No. 5,328,682 discloses mouthwashes containing abrasive silica components to provide for a “rinse and brush” type product. The abrasive silica is maintained in a substantially stable suspension with suspending agents such as smectite and montmorillonite clays, carboxymethylcellulose, xanthan gum or acrylic acid polymers. The abrasive silica functions to clean the tooth surfaces and is meant to be expectorated with the mouthwash. The '682 patent does not address how to deliver silica or silicate materials for long term deposition to the tooth surfaces to provide therapeutic benefit after rinsing and expectorating of the mouthwash. No consideration has been made for the delivery of solid particles from a liquid mouthwash in which the solid particles are to adhere to tooth surfaces for any purpose under typical mouthwash utilization. With the focus on mouthwashes as therapeutically beneficial for certain end-uses, there exists a need to help provide therapeutic benefits other than abrasion through a simultaneous mouth rinsing activity.

### BRIEF SUMMARY AND ADVANTAGES OF THE INVENTION

A mouth rinse composition is provided that permits delivery of particulate materials having affinity for adhesion to the surface of teeth through common mouth rinsing procedures. Such compositions must exhibit proper suspension of particulate materials to prevent settling during storage while simultaneously providing proper mouth rinsing properties. The particulate materials may themselves exhibit any number of therapeutic or aesthetic benefits as long as such materials are easily transferred through mouth rinsing and exhibit proper affinity for deposit on target teeth upon contact therewith. Examples of therapeutic benefit include reduction in tooth sensitivity and remineralization of the tooth surface by application of calcium. The particulate materials, in one embodiment, exhibit certain ionic charge levels as well as sufficiently small particle sizes to permit effective static attraction and eventual accumulation on target tooth surfaces. The particulate materials may be adduct-treated precipitated silica, calcium silicate or a calcium silicate modified by reaction with phosphoric acid.

One distinct advantage of the present inventive mouth rinse composition is the ability to deliver therapeutically beneficial particulate materials from a very low viscosity liquid through maneuvering of the liquid within a person's oral cavity. Another advantage of this invention is the ability to provide the necessarily low viscosity of the overall mouth rinse composition but still suspend the desired particulate materials to the extent that such materials are homogeneously mixed throughout the mouth rinse composition for substantially uniform delivery to target tooth surfaces. Another advantage of this invention is the sufficient degree of affinity with target tooth surfaces exhibited by the specific precipitated silica materials to permit long-term adhesion to such tooth surfaces, allowing for deposition of such materials during typical utilization of mouth rinse compositions. Still another advantage of this invention is the ability to include such silica materials in mouth rinse compositions that either exhibit or are attached to compounds that exhibit therapeutic and/or aesthetic benefits to accord simultaneous silica deposition and typical mouthwash benefits.

Accordingly, this invention encompasses mouth rinse compositions exhibiting a viscosity of at most 10,000 cps (preferably less than 5,000 cps, most preferably between 1 and 2,000 cps) at 25° C. and 1 atmosphere pressure and comprising a base solvent of preferably water, alcohol or mixture thereof, and from 0.01 to 25% by weight of particulate material wherein the particulate material exhibits an affinity for adhesion to tooth surfaces when applied through a standard mouth rinsing procedure and the particulate material does not exhibit appreciable separation or settling from within the mouth rinse compositions after at least 3 weeks of storage at room temperature. In another embodiment, the particulate materials are present in an amount of from 0.01 to about 10% of the total weight of the mouth rinse composition. Preferably, the amount is from 0.05 to about 5% by weight.

Also encompassed within this invention is a method of depositing particulate materials to tooth surfaces through the introduction of such a mouth rinse compositions within the oral cavity of a user, and particularly materials that impart therapeutic benefits (or aesthetic benefits) to target teeth upon such deposition.

A standard mouth rinse procedure is intended to entail the introduction of a certain amount of the mouth rinse composition (such as from 5 to 20 mL, as examples) by pouring the mouth rinse composition into a delivery vessel and transferring the mouth rinse composition to the user's oral cavity. The user would then move the fluid mouth rinse composition around the oral cavity to best ensure contact between the mouth rinse composition and most, if not all, of the different locations within the oral cavity. After a suitable period of time (for example, 10 seconds), the user will then expectorate the mouth rinse composition out of the oral cavity, thereby leaving residual amounts of liquid and other mouth rinse components within the oral cavity. In essence, the mouth rinse procedure encompasses the introduction of the mouth rinse composition within a user's oral cavity for a period of time to coat internal areas of the oral cavity, followed by expectoration thereof.

### DETAILED DESCRIPTION OF THE INVENTION

All parts, percentages and ratios used herein are expressed by weight unless otherwise specified. All documents cited herein are incorporated by reference.

Since mouth rinse compositions are liquid in nature, and thus exhibit a viscosity of at most 10,000 cps (as noted above), the starting point for such compositions are the base solvents. Typically, water can be the main ingredient to permit the necessary liquid form as well as for cost reasons. Alcohols may also be utilized, specifically for their ability to either kill microorganisms (such as ethanol) or provide aesthetic properties to the overall composition (such as menthol). However, for evident reasons, the alcohol present should be non-toxic in nature. The water/alcohol (or alcohol alone or water alone) portion should be the vast majority of the mouth rinse composition of the present invention in order to, again provide the desired liquid characteristics for ease in transfer, delivery, and removal from the user's oral cavity. Thus, the base solvent may constitute from 75-99.99% by weight of the overall formulation.

More importantly, though, this mouth rinse composition includes particulate materials that exhibit an affinity for adhesion to tooth surfaces when applied through standard mouth rinsing procedure. The particulate materials may be selected from precipitated silica, silicate or mixtures thereof. In some instances the precipitated silica is modified to permit deposition on target tooth surfaces during a mouth rinsing procedure. This modification generates silica that will easily adhere to tooth surfaces as compared to the same material in a non-modified state which would typically not adhere to tooth surfaces. As disclosed in co-pending U.S. application Ser. No. 12/499,359, filed Jul. 8, 2009 (“the '359 application”), tubule-blocking precipitated silica materials, which exhibit a greater propensity to adhere to tooth surfaces, were introduced within a dentifrice formulation and through a typical tooth brushing process. These same silica materials have now been found to be useful particulate materials for application within the mouth rinse compositions of the present invention. U.S. Ser. No. 12/499,359 is herein incorporated in its entirety.

While any type of modification of silica that accords an adhesion increase on tooth and gum surfaces is included within this invention, the modifications disclosed in the '359 application provide for one of the preferred embodiments of the present invention. These modifications in the '359 application generate a precipitated silica material having i) a mean particle size of 1 to 5 microns and ii) an adduct present on at least a portion of the surface of the material to form an adduct-treated precipitated silica material that exhibits a zeta potential reduction of greater than 10% of the zeta potential of a similar precipitated silica material on which no adduct is present. In one embodiment, the adduct is a metal element. In another embodiment, the adduct is a metal element selected from the transition metals and post-transition metals. Examples of suitable metal elements include aluminum, zinc, tin, strontium, iron, copper, and mixtures thereof. The adduct-treated precipitated silica material is formed by the addition of the adduct in the form of a water-soluble metal salt during the formation of precipitated silica material, which is further detailed below. Any metal salt that is soluble in acidic conditions would be suitable, such as metal nitrates, metal chlorides, metal sulfates, and the like.

In one embodiment, the adduct-treated precipitated silica material exhibits a zeta potential reduction greater than 15% when compared to a precipitated silica material of the same structure on which no adduct is present. In another embodiment, the zeta potential reduction is greater than 20%. In still another embodiment, the zeta potential reduction is greater than 25%.

Other types of silica modification that exhibit increased affinity for bovine dentin are cationic in nature or are surface enriched with minor metal oxide components. Metal stabilized colloidal silicas, such as those manufactured under the trade name Ludox by W.R. Grace, particularly the Ludox AM series, are typically 5 to 120 nm in size and can be treated with an aluminum species or other metal component. This treatment results in a reduction of the negative charge of the silica or the formation of cationically charged particles depending on the composition and level of treatment of the silica, pH and the ionic strength of the colloidal dispersion. Mixed metal co-fumed silica products, such as those produced by addition of a small amount of a minor metal halogen stream during the pyrogenic silica manufacturing process, are approximately 300 nm in size when dispersed in aqueous solution. Addition of the small amounts of the minor metal salt results in a surface enrichment yielding a reduction of the negative surface charge of silica or a cationic particle dependent on the composition and level of treatment, pH and the ionic strength of the dispersion. Such commercial products are sold under the tradename Aerosil MOX 80 or MOX 170 (available from Evonik). Both the colloidal silicas and co-fumed metal oxide products are available commercially in the form of a slurry, which could be conveniently added to the mouthwash formulations described herein to form mouth rinse compositions of the present invention. Although these particles are much smaller in size than bovine tubules, their modified surface charge should enable them to interact with bovine dentin with a greater affinity when compared to non-surface enriched colloidal and fumed silica particles alone.

For silicate materials, one preferred embodiment is that of calcium silicate which may have similarly sized particles as that of the '359 application. Additionally, calcium silicate may be modified by reaction with phosphoric acid to generate a calcium phosphate salt, which aids in allowing for ionic charges to adhere the calcium silicate to the tooth surfaces, or within the dentinal tubules of teeth, or even on gum tissue surfaces. Such calcium silicate/phosphate compounds can thus deliver calcium ions to adhered-to teeth for potential remineralization purposes.

In the '359 application, the modified precipitated silica materials are added to dentifrices which typically include silica materials for either abrasive effect or thickening characteristics, not for delivery of other compounds or for the ability of such silica or silicate particles to provide other benefits. However, the presence of other types of components within a dentifrice may potentially deleteriously affect deposition of modified silica or silicate particulates, or possibly modified silica or silicate particulates themselves may deleteriously affect certain dentifrice compounds (fluoride sources, for example, may react undesirably with certain metallic compounds thereby rendering the fluoride unavailable for utilization). Thus, it was important to first realize that silica or silicate materials may offer promise to deliver certain benefits to tooth and gum surfaces beyond those provided within typical dentifrice compositions. It has been now realized that silica and silicate materials may be coupled with breath fresheners (for example, “stored” within the pores of the silica or silicate materials for delayed, but continuous delivery over time), coupled with calcium sources (for remineralization purposes), and used as actual tubule blocking components (to reduce discomfort to a user due to accessible dentinal tubules). The problem with such a potential capability was the delivery, reliably, of such materials to tooth surfaces in a manner other than through typical dentifrice formulations and tooth brushing procedures.

Once the proper silica or silicate particulate and their modifications was realized for affinity to tooth surfaces, the next challenge was to formulate mouth rinse compositions that would have proper suspension while stored, proper transfer to a user's oral cavity, proper deposition on a user's tooth and gum tissue surfaces during a mouth rinsing procedure, and delivery of desired benefits thereafter to the tooth and gums of the user. The difficulty encountered with the potential inclusion of such materials within mouth rinse compositions pertains to the ability to ensure uniform delivery to the user's oral cavity and hoped-for even distribution throughout the tooth surfaces and gum tissues therein. A possible production of thickened mouth rinse compositions was determined to be improper as mouthwash users do not generally want a gelled or creamy mouth rinse; thus a liquid rinse is necessary. The goal was to develop a manner of not only delivering, reliably, silica or silicate particulate materials to tooth surfaces from liquid sources, but also to provide a formulation that did not settle to the bottom of a storage container and required thorough mixing to at least move the settled particulates to the top for transfer.

To that end, certain polysaccharide gums were found to provide suitable hydration levels in low proportions with effective suspension characteristics. Xanthan gum, high acetyl gellan gum, and low acetyl gellan gum exhibited excellent capability for such a purpose. Additionally, numerous other types of thickening agents, such as carboxymethylcellulose, or combinations of thickening agents may also provide such beneficial results. Below are greater details in terms of the utilization and preparation of the proper suspensions for particulate material introduction within liquid mouth rinse compositions of the present invention. The gums are generally added in a pre-mix formulation at very low levels (from 0.01 to 0.25% by weight thereof) to accord the necessary suspension properties while still allowing for the overall mouth rinse composition to remain in suitable liquid form.

Other components that may be added to the mouth rinse compositions include typical antimicrobial compounds, surfactants (to aid in deposition of organic liquids to target teeth and oral tissues), sweeteners, flavorants, colorants, and other compounds that permit aesthetic and different potential therapeutic benefits from the particulate materials themselves (such as humectants, preservatives, and the like). Examples of suitable antimicrobial agents to be employed in the mouth rinse compositions include phenolic compounds such as thymol, chlorothymol, amyl-, hexyl-, heptyl- and octylphenol, hexylresorcinol, hexachlorophene, and phenol; quaternary ammonium compounds such as quaternary morpholinium alkyl sulfates, cetylpyridinium chloride, alkyldimethyl benzylammonium chloride, and alkyltrimethyl ammonium halides; and miscellaneous antibacterial compounds such as benzoic acid, formaldehyde, potassium chlorate, tyrothricin, gramicidin, iodine, sodium perborate, and urea peroxide. |As|_{[A1]} well, sodium benzoate may be included as well to dissociate into benzoic acid for such a purpose. Weak acids may be added as well as buffering agents to adjust the pH levels. Thus, citric acid, tartaric acid, and acetic acid may also be added. Exemplary buffering agents are an alkali metal or alkaline earth metal salt, and an amine (e.g., ammonium) salt of the weak carboxylic acid. The preferred buffering agents are sodium citrate, potassium citrate, and sodium acetate. Surfactants may be included in the composition to keep the mouth rinse compositions clear and to prevent turbidity. Any food-grade surfactants can be employed for this purpose, such as anionic alkyl sulfates (sodium lauryl sulfate, sodium tetradecyl sulfate, and the like). Sweetening agents such as sodium saccharin, sorbitol, xylitol, aspartame, and sucrose may also be included. The flavorants can be selected from cinnamon, cassia, anise, menthol, eucalyptol, methyl salicylate, peppermint oil, spearmint oil, and other known flavor modifiers. Colorants, such as FD&C dyes, may be added as well. These extra components may be present from 0.01 to 25% of the total mouth rinse composition by weight.

The precipitated silica materials of the present invention are prepared according to the following process. An aqueous solution of an alkali silicate, such as sodium silicate, is charged into a reactor equipped with mixing means adequate to ensure a homogeneous mixture. The alkali silicate solution in the reactor is preheated to a temperature of between about 65° C. and about 100° C. The alkali silicate aqueous solution may have an alkali silicate concentration of approximately 8.0 to 35 wt %, such as from about 8.0 to about 20 wt %. The alkali silicate may be a sodium silicate with a SiO2:Na2O ratio of from about 1 to about 3.5, such as about 2.4 to about 3.4. The quantity of alkali silicate charged into the reactor is about 5 wt % to 100 wt % of the total silicate used in the batch. Optionally, an electrolyte, such as sodium sulfate solution, may be added to the reaction medium. Additionally, this mixing may be performed under high-shear conditions.

To the reactor is then simultaneously added: (1) an aqueous solution of an acidulating agent or acid, such as sulfuric acid; and (2) additional amounts of an aqueous solution containing the same species of alkali silicate as is in the reactor, the aqueous solution being preheated to a temperature of about 65° C. to about 100° C. An adduct compound is added to the acidulating agent aqueous solution prior to the introduction of the acidulating agent aqueous solution into the reactor. The adduct compound is premixed with the acidulating agent aqueous solution in a concentration of mol. of adduct compound to L of acidulating agent aqueous solution of about 0.002 to about 0.185, preferably about 0.074 to about 0.150. Optionally, if higher adduct concentrations are required in the final silica product, an aqueous solution of the adduct compound can be used in place of the acid.

The acidulating agent solution preferably has a concentration of acidulating agent of about 6 to 35 wt %, such as about 9.0 to about 20 wt %. After a period of time the inflow of the alkali silicate is stopped and the acidulating agent is allowed to flow until the desired pH is reached.

The reactor batch is allowed to age or “digest” for between 5 minutes to 30 minutes at a set digestion temperature, with the reactor batch being maintained at a constant pH. After the completion of digestion, the reaction batch is filtered and washed with water to remove excess by-product inorganic salts until the wash water from the silica filter cake obtains a conductivity of less than about 2000 μmhos/cm. Because the conductivity of the silica filtrate is proportional to the inorganic salt by-product concentration in the filter cake, then by maintaining the conductivity of the filtrate to be less than 2000 μmhos, the desired low concentration of inorganic salts, such as Na₂SO₄ in the filter cake may be obtained. The silica filter cake is slurried in water, and then dried by any conventional drying techniques, such as spray drying, to produce adduct-treated precipitated silica material containing from about 3 wt % to about 50 wt % of moisture. The adduct-treated precipitated silica material may then be milled to obtain the desired particle size of between about 1 μm to 5 μm. The adduct-treated precipitated silica material mentioned above is preferably a water-soluble metal salt that when added to the silica reduces the negative charge on the silica. Examples of suitable metal elements include elements selected from transition and main group elements. Preferred examples of the water-soluble metal salt include aluminum, zinc, tin, strontium, iron, and copper.

Calcium silicates are most typically prepared by the reaction of reactive silica with an alkaline earth metal reactant, preferably an alkaline earth metal oxide or hydroxide, and a source of aluminum such as sodium aluminate or alumina. Because the final properties of the silicate are dependent on the reactivity of the silica, the silica source is preferred to be the reaction product of a soluble silicate, such as sodium silicate, and a mineral acid, such as sulfuric acid. Suitable synthetic amorphous alkaline earth metal silicates are manufactured by the J. M. Huber Corporation and are sold in different grades under the trademark Hubersorb®. Methods and techniques for preparing these silicas are discussed in greater detail in U.S. Pat. No. 4,557,916, which is herein incorporated in its entirety. In one embodiment, a calcium silicate is reacted with phosphoric acid to form a modified calcium silicate/phosphate for adherence to target tooth surfaces.

Such particulate silica or silicate materials may be introduced as prepared for the purpose of occluding dentinal tubules. Alternatively, such materials may then be reacted with other compounds to utilize the silica or silicate as deposition agents for delivery of the other compounds to the tooth and/or gum tissue surfaces. Thus, for instance, the pores of such silica or silicate materials may be filled with various agents, such as breath freshening compounds, to permit delivery over time within the user's oral cavity. Likewise, a calcium source may be added (such as calcium silicate reacted with phosphoric acid, noted above) to provide a remineralization compound to target tooth surfaces. In such a manner, the calcium portion of the delivered compound may react with the tooth to permit reconstitution of any lost calcium from the tooth tissues over time.

### BRIEF DESCRIPTION OF THE DRAWINGS

FIG. 1 is a series of photomicrographs showing the results of a synthesized mouthwash affinity test of two suspended samples in terms of occlusion capability of modified silica according to the invention as compared with non-modified silica within dentinal tubules from a xanthan gum suspension system.

FIG. 2 is a series of photomicrographs showing the results of a store-bought mouthwash affinity test of a sample in terms of occlusion capability of modified silica as compared with non-modified silica within dentinal tubules from a xanthan gum suspension system.

FIG. 3 is a series of photomicrographs showing the results of a synthesized mouthwash affinity test of two suspended samples in terms of occlusion capability of modified silica as compared with non-modified silica within dentinal tubules from a high acyl gellan gum suspension system.

FIG. 4 is a series of photomicrographs showing the results of a store-bought mouthwash affinity test of a sample in terms of occlusion capability of modified silica as compared with non-modified silica within dentinal tubules from a high acyl gellan gum suspension system.

FIG. 5 is a series of photomicrographs showing the results of a synthesized mouthwash affinity test of two suspended samples in terms of occlusion capability of modified silica as compared with non-modified silica within dentinal tubules from a low acyl gellan gum suspension system.

FIG. 6 is a series of photomicrographs showing the results of a store-bought mouthwash affinity test of a sample in terms of occlusion capability of modified silica as compared with non-modified silica within dentinal tubules from a low acyl gellan gum suspension system.

FIG. 7 is a series of photomicrographs showing the results of a synthesized mouthwash affinity test of two suspended samples in terms of tooth surface adherence of calcium silicate reacted with phosphoric acid and calcium silicate from a xanthan gum suspension system.

### PREFERRED EMBODIMENTS OF THE INVENTION

Without intending on any limitation of the breadth of scope of this invention, provided are preferred examples of certain aspects thereof.

Initially, determinations of proper suspensions of silica materials in liquid formulations were necessary. For these purposes, two different types of silica particulates were provided. ZEOTHIX® 177 is a precipitated silica thickener having an average particle size of approximately 3.5 microns (available from J.M. Huber Corporation). ZEODENT® 113 is a precipitated silica dental abrasive having an average particle size of approximately 10 microns as commercially supplied and was then air milled to an average particle size of approximately 3 microns (available from J.M. Huber Corporation). When ˜10 g of each silica material was added to a beaker of 190 g of deionized water and stirred at 1000 rpm for 20 minutes, the materials all settled to the bottom of the beaker within a short period of time (3 minutes at the most, generally) once stirring was stopped.

Samples incorporating KELDENT® xanthan gum (available from CP Kelco U.S., Inc. of Atlanta, Ga.), KELCOGEL® gellan gum (available from CP Kelco U.S., Inc. of Atlanta, Ga.), and KATHON™ CG preservative (available from Rohm & Haas, which is used herein solely for experimental purposes in terms of determining settling characteristics of tested samples and is generally not included within commercial mouthwashes) were made in accordance with the following tables and the procedures provided thereafter:

TABLE 1
KELDENT ® Xanthan Gum and Silica in Water

| | Formulation 1 | | Formulation 2 | |
|---|---|---|---|---|
| | Weight | Quantity | Weight | Quantity |
| Ingredient | (%) | (g) | (%) | (g) |
| Zeodent ® 113 silica | 5.0 | 10.0 | — | — |
| abrasive (air milled) | | | | |
| Zeothix ® 177 silica | — | — | 5.0 | 10.0 |
| thickener | | | | |
| Keldent ® Xanthan Gum | 0.2 | 0.4 | 0.2 | 0.4 |
| Kathon ™ CG | 0.1 | 0.2 | 0.1 | 0.2 |
| preservative | | | | |
| Distilled water | q.s. to 100 | 189.4 | q.s. to 100 | 189.4 |

With regard to Table 1, Formulation 1 was prepared with 0.4 g of KELDENT® xanthan gum added to a beaker containing 189.4 g of deionized water and was stirred at approximately 1000 rpm for 20 minutes. Once the xanthan gum was dispersed and hydrated, 10.0 g of air milled ZEODENT® 113 was slowly added and the solution was mixed for 10 minutes. After mixing, 0.2 g of Kathon™ CG was added as a preservative. The same procedure was used to prepare a Formulation 2, with the exception ZEOTHIX® 177 was used in place of ZEODENT® 113.

TABLE 2
KELCOGEL ® CG-HA Gellan Gum and Silica in Water

| | Formulation 3 | | Formulation 4 | |
|---|---|---|---|---|
| | Weight | Quantity | Weight | Quantity |
| Ingredient | (%) | (g) | (%) | (g) |
| Zeodent ® 113 silica | 5.0 | 10.0 | — | — |
| abrasive (air milled) | | | | |
| Zeothix ® 177 silica | — | — | 5.0 | 10.0 |
| thickener | | | | |
| Kelcogel ® CG-HA | 0.05 | 0.1 | 0.05 | 0.1 |
| Gellan Gum | | | | |
| Kathon ™ CG | 0.1 | 0.2 | 0.1 | 0.2 |
| preservative | | | | |
| Distilled water | q.s. to 100 | 189.4 | q.s. to 100 | 189.4 |

With regard to Table 2, Formulation 3 was prepared with 0.1 g of KELCOGEL® CG-HA Gellan Gum (high acyl) added to a beaker containing 189.4 g of deionized water and was stirred at approximately 1000 rpm at 85° C. for 20 minutes. Once the gellan gum was dispersed and hydrated, 10.0 g of air milled ZEODENT® 113 was slowly added and to the solution with continued stirring. The solution was gradually allowed to cool to room temperature with stirring. 0.2 g of Kathon™ CG was then added as a preservative. The same procedure was used to prepare a Formulation 4, with the exception ZEOTHIX® 177 was used in place of ZEODENT® 113.

TABLE 3
Kelcogel ® CG-LA Gellan Gum and Silica in Water

| | Formulation 5 | | Formulation 6 | |
|---|---|---|---|---|
| | Weight | Quantity | Weight | Quantity |
| Ingredient | (%) | (g) | (%) | (g) |
| Zeodent ® 113 silica | 5.0 | 10.0 | — | — |
| abrasive (air milled) | | | | |
| Zeothix ® 177 silica | — | — | 5.0 | 10.0 |
| thickener | | | | |
| Kelcogel ® LA Gellan | 0.05 | 0.1 | 0.05 | 0.1 |
| Gum | | | | |
| Kathon ™ CG- | 0.1 | 0.2 | 0.1 | 0.2 |
| preservative | | | | |
| Distilled water | q.s. to 100 | 189.4 | q.s. to 100 | 189. |

With regard to Table 3, Formulation 5 was prepared with 0.1 g of Kelcogel CG-LA (low acyl) added to a beaker containing 189.4 g of deionized water and was stirred at approximately 1000 rpm at 85° C. for 20 minutes. Once the gellan gum was dispersed and hydrated, 10.0 g of air milled Zeodent® 113 was slowly added and to the solution with continued stirring. The solution was gradually allowed to cool to room temperature with stirring. 0.2 g of Kathon™ CG was then added as a preservative. The same procedure was used to prepare Formulation 6, with the exception ZEOTHIX® 177 was used in place of ZEODENT® 113.

None of the Formulations 1-6 exhibited any noticeable settling of the silica particulates after one week of storage. Thus, it was decided to utilize a similar base system for inclusion within a mouth rinse composition to suspend solid particles. Other silica materials were tested in mouth rinse compostions for suspension properties. ZEODENT® 165 is precipitated silica having an average particle size of 12 microns after hammer milling (available from J.M. Huber Corporation). ZEOTHIX® 265 is precipitated silica having an average particle size of approximately 3 microns after air milling and containing approximately 0.5% alumina (available from J.M. Huber Corporation). PLURONIC® F127 is a surfactant available from BASF Corporation.

### Modified Particulate Materials:

Initially, an adduct-treated precipitated silica material was produced via the following process:

67 L of silicate (19.5%, 1.180 g/mL, 3.32 MR) and 167 L of water were added to the 400 gallon reactor and heated to 87° C. with recirculation at 30 HZ and stirring at 60 RPM. Silicate (19.5%, 1.180 g/mL, 3.32 MR) and a sulfuric acid/alum solution (17.1%, 1.12 g/mL sulfuric acid containing 0.22 mol Alum/L acid) were then simultaneously added at 12.8 L/min and 1.2 L/min for 47 minutes. After 47 minutes, the flow of silicate was stopped and the pH was adjusted to 5.5 with continued flow of acid. Once pH 5.5 was reached, the batch was allowed to digest for 10 minutes and was then dropped. It was filtered and washed to a conductivity of ˜1500 μS and was spray dried. A portion of this batch was then air milled to an average particle size of ˜3.0 μm. This adduct-treated precipitated silica was tested for various properties, in accordance with the following test protocols.

Metal levels in the silica were made by standard elemental analysis techniques. The silica was dissolved in HClO₄ and HF and was then heated to drive off all volatile components. The remaining non-volatiles were then dissolved in dilute HCl and their concentrations determined by analysis on a Perkin Elmer Optima 3000 ICP/OES.

Moisture or Loss on Drying (LOD) was the measured silica sample weight loss of 2.0 g of sample at 105° C. for 2 hours.

A conductivity method was used to measure sodium sulfate concentrations within the final silica products. A 5% slurry of silica in deionized water was prepared and the conductivity measured with a conductivity meter. This was then related back to the % sodium sulfate by a correlation table. In the plant or pilot plant, typically the filtrate was measured from rotary or plate and frame filter and washing parameters adjusted to target a conductivity of less than 2000 μmho/cm.

The CTAB external surface area of silica was determined by adsorption of CTAB (cetyltrimethylammonium bromide) on the silica surface, the excess separated by centrifugation and determined by titration with sodium lauryl sulfate using a surfactant electrode. The external surface of the silica was determined from the quantity of CTAB adsorbed (analysis of CTAB before and after adsorption). Specifically, about 0.5 g of silica was accurately weighed and placed in a 250-ml beaker with 100.00 ml CTAB solution (5.5 g/L, adjusted to pH 9.0±0.2), mixed on an electric stir plate for 30 minutes, then centrifuged for 15 minutes at 10,000 rpm. 1.0 ml of 10% Triton X-100 is added to 5.0 ml of the clear supernatant in a 100-ml beaker. The pH was adjusted to 3.0-3.5 with 0.1 N HCl and the specimen was titrated with 0.0100 M sodium lauryl sulfate using a surfactant electrode (Brinkmann SUR15O1-DL) to determine the endpoint.

The oil absorption values were measured using the rubout method. This method is based on a principle of mixing linseed oil with silica by rubbing with a spatula on a smooth surface until a stiff putty-like paste is formed. By measuring the quantity of oil required to have a paste mixture which will curl when spread out, one can calculate the oil absorption value of the silica—the value which represents the volume of oil required per unit weight of silica to saturate the silica sorptive capacity. A higher oil absorption level indicates a higher structure of precipitated silica; similarly, a low value is indicative of what is considered a low-structure precipitated silica. Calculation of the oil absorption value was done as follows:

$$\begin{matrix}Oil absorption = \frac{ml oil absorbed}{weight of silica , grams} \times 100 \\ = ml oil \text{/} 100 gram silica\end{matrix}$$

Median particle size was determined using a Model LA-930 (or LA-300 or an equivalent) laser light scattering instrument available from Horiba Instruments, Boothwyn, Pa.

The % 325 mesh residue of silica was measured utilizing a U.S. Standard Sieve No. 325, with 44 micron or 0.0017 inch openings (stainless steel wire cloth) by weighing a 10.0 gram sample to the nearest 0.1 gram into the cup of a 1 quart Hamilton mixer Model No. 30, adding approximately 170 ml of distilled or deionized water and stirring the slurry for at least 7 min. The mixture was transferred onto the 325 mesh screen and water was sprayed directly onto the screen at a pressure of 20 psi for two minutes, with the spray head held about four to six inches distant from the screen. The remaining residue was then transferred to a watch glass and dried in an oven at 150° C. for approx. 15 min.; then cooled and weighed on an analytical balance.

The pH values of the reaction mixtures (5 weight % slurry) can be monitored by any conventional pH sensitive electrode.

To measure brightness, samples were pressed into a smooth surfaced pellet and evaluated with a Technidyne Brightmeter S-5/BC. This instrument has a dual beam optical system where the sample is illuminated at an angle of 45°, and the reflected light viewed at 0°.

For the adduct-treated precipitated silica (Silica-Al) produced above, measurements of properties were undertaken and are provided in Tables A and B. The alumina content was 2% by weight.

TABLE A
Metal Oxide Amounts

| Al₂O₃ | CaO | Fe₂O₃ | MgO | Na₂O | TiO₂ |
|---|---|---|---|---|---|
| (ppm) | (ppm) | (ppm) | (ppm) | (ppm) | (ppm) |
| 19600 | 37 | 141 | 79 | 1.48 | 131 |

TABLE B
Silica Properties

| | Sodium | 325 | | |
|---|---|---|---|---|
| Moisture | Sulfate | residue | BET | CTAB |
| (%) | (%) | (%) | (m²/g) | (m²/g) |
| 5.7 | <0.35 | 0.00 | 349 | 68 |

| Mean | Median | Oil | | |
|---|---|---|---|---|
| Particle | Particle | Absorption | 5% | |
| Size (μm) | Size (μm) | (cc/100 g) | pH | Brightness |
| 3.6 | 3.5 | 89 | 8.2 | 99.0 |

Zeta potential is a measure of the charge on the external surface of a particle suspended in solution. Particles with zeta potentials of the same charge will tend to repel one another and particles with zeta potentials of the opposite charge will tend to be attracted to one another. Historically, zeta potential has been determined by microelectrophoresis, whereby an electric field is applied across a dispersion of particles and the velocity of the particles as they migrate toward an electrode of opposite charge is measured. Particles traveling at a greater velocity toward the electrode of opposite charge will tend to have an increased magnitude of charge on their surface. Alternatively, zeta potential can be determined by an electrokinetic sonic amplitude (ESA) technique. ESA measures the electrokinetic properties of a particle by an electroacoustic method. A high frequency oscillating electric field is applied to a dispersion of particles. The particles will oscillate with the applied field proportional to the charge on their surface. As the particles move in one direction, the liquid they displace will move in the other. If there are density differences between the particles and the liquid medium, an acoustic wave will be generated at the interface of the electrode and the liquid dispersion as a result of the liquid that is displaced by the moving particles. The acoustic wave generated can then be measured and the intensity of the wave is then related to the magnitude of the zeta potential. Zeta potential is usually measured across a range of pH values, thus giving an indication of how the surface charge of the suspended particles varies as a function of pH (Greenwood, R. “Review of the measurement of zeta potentials in concentration aqueous suspensions using electroacoustics” Advances in Colloid and Interface Science, 2003, 106, 55-81, herein entirely incorporated by reference).

The zeta potential of the adduct-treated precipitated silica (Silica-Al) produced above was measured and compared to the measurement of a similar precipitated silica not containing the adduct. The percent reduction in zeta potential was 29.16% at a pH of 8.0.

Also tested were calcium silicate (HUBERSORB® 600 available from J.M. Huber Corporation) and a calcium silicate modified by reaction with phosphoric acid (CA Silicate-PA). This modified calcium silicate was produced as follows:

5 g of HUBERSORB® 600 was stirred in a 500 ml beaker containing 200 g of deionized water. Concentrated phosphoric acid was added drop wise until the pH dropped from 9.6 to approximately 7.0. Additional acid was added over the next 60 minutes as needed to keep the pH between 6.5 and 7.0. After 60 minutes, the silicate was filtered and dried overnight at 105° C. It was milled in a laboratory sized coffee mill to break up large particles that formed as a result of drying. This CA Silicate-PA was then analyzed for certain physical and chemical properties as well. Calcium and phosphate percentages were undertaken in typical fashion. The remaining measurements were made in accordance with those noted above.

TABLE C
Calcium Silicate Properties

| | | | | Median | | | |
|---|---|---|---|---|---|---|---|
| | H₂O | 5% | Oil Absorption | particle | 325 mesh | Ca | P |
| Sample | (%) | pH | (cc/100 g) | size (μm) | residue (%) | (%) | (%) |
| Calcium silicate | 6.0 | 9.8 | 458 | 7.3 | 0.53 | — | — |
| CA Silicate-PA | — | — | — | — | — | 15.1 | 8.8 |

All of the above-noted samples were then incorporated into mouth rinse compositions in accordance with the tables and procedures below (all amounts are in grams unless otherwise noted):

### Silica Samples in Mouthwash

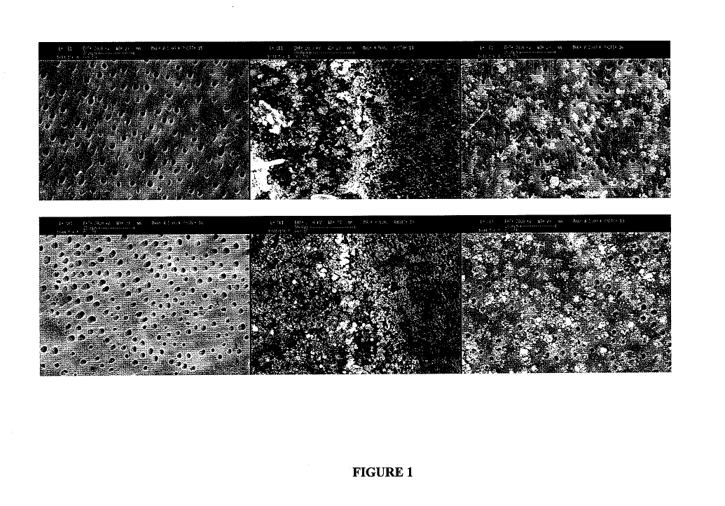

In the following Tables 4-11 and FIGS. 1-7, examples representative of the present invention are Example 5, Example 11, Example 17, Example 23, Example 29, Example 35, Example 37 and Example 38. All other examples represent comparative examples.

TABLE 4
Keldent ® Xanthan Gum and Silica in Synthesized Mouthwash

| Ingredient in | Mouthwash | | | | | | |
|---|---|---|---|---|---|---|---|
| grams | Base 1 | Ex. 1 | Ex. 2 | Ex. 3 | Ex. 4 | Ex. 5 | Ex. 6 |
| Ethyl Alcohol | 227.0 | — | — | — | — | — | — |
| Pluronic F127 | 5.0 | — | — | — | — | — | — |
| Sodium | 1.5 | — | — | — | — | — | — |
| benzoate | | | | | | | |
| Keldent | 2.0 | — | — | — | — | — | — |
| xanthan gum | | | | | | | |
| Deionized | 461.6 | — | — | — | — | — | — |
| water | | | | | | | |
| Citric acid | 0.1 | — | — | — | — | — | — |
| Sodium citrate | 0.3 | — | — | — | — | — | — |
| 70% Sorbitol | 250.0 | — | — | — | — | — | — |
| solution | | | | | | | |
| Mouthwash | — | 39.0 | 39.0 | 39.0 | 39.0 | 39.0 | 39.0 |
| Base 1 | | | | | | | |
| Zeothix 177 | — | 1.0 | — | — | — | — | — |
| Zeothix 265 | — | — | 1.0 | — | — | — | — |
| Zeodent 165 | — | — | — | 1.0 | — | — | — |
| Zeodent 113 | — | — | — | — | 1.0 | — | — |
| Silica-Al | — | — | — | — | — | 1.0 | — |

With regard to Table 4, a pre-mix (mouthwash base) was prepared by adding 5.0 g Pluronic F127 and 1.5 g sodium benzoate to a 1500 ml beaker containing 227.0 g of ethyl alcohol (95%). The solution was stirred for approximately 30 minutes to dissolve the surfactant and salt. 461.6 g of deionized water, 250.0 g of 70% sorbitol and 2.0 g Keldent® xanthan gum were then added and the solution was stirred for approximately 75 minutes to disperse and hydrate the gum. To prepare the individual silica samples, 39.0 g of pre-mix was added to a 50 ml screw top vial containing 1.0 g of silica and it was then stirred for approximately 2.5 hours.

TABLE 5
Keldent ® Xanthan Gum and Silica in Store-Bought Mouthwash

| Ingredient in | Mouthwash | | | | Ex. | Ex. | Ex. |
|---|---|---|---|---|---|---|---|
| grams | Base 2 | Ex. 7 | Ex. 8 | Ex. 9 | 10 | 11 | 12 |
| LISTERINE ® | 1000.0 | — | — | — | — | — | — |
| Fresh Burst | | | | | | | |
| Mouthwash | | | | | | | |
| Keldent | 2.0 | — | — | — | — | — | — |
| xanthan gum | | | | | | | |
| Mouthwash | — | 39.0 | 39.0 | 39.0 | 39.0 | 39.0 | 39.0 |
| Base 2 | | | | | | | |
| Zeothix 177 | — | 1.0 | — | — | — | — | — |
| Zeothix 265 | — | — | 1.0 | — | — | — | — |
| Zeodent 165 | — | — | — | 1.0 | — | — | — |
| Zeodent 113 | — | — | — | — | 1.0 | — | — |
| Silica-Al | — | — | — | — | — | 1.0 | — |

With regard to Table 5, a pre-mix was prepared by adding 2.0 g of Keldent® xanthan gum to 1000 g of Fresh Burst Listerine. The mixture was stirred for 2 hours to disperse and hydrate the xanthan gum. After two hours of stirring, 39.0 g of the pre-mix was added to a 50 ml screw top vial containing 1.0 g of silica. It was then stirred for approximately two hours.

TABLE 6
Kelcogel ® GC-HA Gellan Gum and
Silica in Synthesized Mouthwash

| Ingredient in | Mouthwash | Ex. | Ex. | Ex. | Ex. | Ex. | Ex. |
|---|---|---|---|---|---|---|---|
| grams | Base 3 | 13 | 14 | 15 | 16 | 17 | 18 |
| Ethyl Alcohol | 250.0 | — | — | — | — | — | — |
| Pluronic F127 | 5.0 | — | — | — | — | — | — |
| Sodium | 1.5 | — | — | — | — | — | — |
| benzoate | | | | | | | |
| Kelcogel GC- | 0.47 | — | — | — | — | — | — |
| HA gellan gum | | | | | | | |
| Deionized | 461.6 | — | — | — | — | — | — |
| water | | | | | | | |
| Citric acid | 0.1 | — | — | — | — | — | — |
| Sodium citrate | 0.3 | — | — | — | — | — | — |
| 70% Sorbitol | 250.0 | — | — | — | — | — | — |
| solution | | | | | | | |
| Mouthwash | — | 39.0 | 39.0 | 39.0 | 39.0 | 39.0 | 39.0 |
| Base 3 | | | | | | | |
| Zeothix 177 | — | 1.0 | — | — | — | — | — |
| Zeothix 265 | — | — | 1.0 | — | — | — | — |
| Zeodent 165 | — | — | — | 1.0 | — | — | — |
| Zeodent 113 | — | — | — | — | 1.0 | — | — |
| Silica-Al | — | — | — | — | — | 1.0 | — |

With regard to Table 6, a pre-mix was prepared by adding 250 g 70% sorbitol, 1.5 g sodium benzoate, 0.1 g citric acid and 0.3 g sodium citrate to a 1500 ml beaker containing 461.6 g of deionized water. The solution was stirred until the salts dissolved. Separately, 5.0 g of Pluronic F127 was dissolved in 227.0 g of 95% ethanol with stirring. After the surfactant was completely dissolved, the two solutions were combined. The resulting solution became cloudy, but cleared up after a few minutes of stirring. 0.47 g of Kelcogel® CG-HA was added and the solution was heated to 75° C. with stirring. Once the temperature reached 75° C., the solution was removed from heat. While the solution was still hot, 39.0 g of the pre-mix solution was added to a 50 ml screw top vial containing 1.0 g of silica and it was then stirred. After approximately 10 minutes of stirring, 0.04 g of sodium chloride was added and the solution was stirred for an additional 2.5 hours.

TABLE 7
Kelcogel ® GC-HA Gellan Gum and
Silica in Store-Bought Mouthwash

| Ingredient in | Mouthwash | Ex. | Ex. | Ex. | Ex. | Ex. | Ex. |
|---|---|---|---|---|---|---|---|
| grams | Base 4 | 19 | 20 | 21 | 22 | 23 | 24 |
| LISTERINE ® | 1000.0 | — | — | — | — | — | — |
| Fresh Burst | | | | | | | |
| Mouthwash | | | | | | | |
| Kelcogel CG- | 0.5 | — | — | — | — | — | — |
| HA gellan gum | | | | | | | |
| Mouthwash | — | 39.0 | 39.0 | 39.0 | 39.0 | 39.0 | 39.0 |
| Base 4 | | | | | | | |
| Zeothix 177 | — | 1.0 | — | — | — | — | — |
| Zeothix 265 | — | — | 1.0 | — | — | — | — |
| Zeodent 165 | — | — | — | 1.0 | — | — | — |
| Zeodent 113 | — | — | — | — | 1.0 | — | — |
| Silica-Al | — | — | — | — | — | 1.0 | — |

With regard to Table 7, a pre-mix was prepared by adding 0.5 g of Kelcogel® CG-HA gellan gum to 1000 g of Fresh Burst Listerine. The solution was stirred for approximately 15 minutes and was then heated with continued stirring to 75° C. Once 75° C. was reached, the solution was removed from heat. While the solution was still hot, 39.0 g of the pre-mix solution was added to a 50 ml screw top vial containing 1.0 g of silica and it was then stirred. After approximately 10 minutes of stirring, 0.04 g of sodium chloride was added and the solution was stirred for an additional 2.5 hours.

TABLE 8
Kelcogel ® GC-LA Gellan Gum and
Silica in Synthesized Mouthwash

| Ingredient in | Mouthwash | Ex. | Ex. | Ex. | Ex. | Ex. | Ex. |
|---|---|---|---|---|---|---|---|
| grams | Base 5 | 25 | 26 | 27 | 28 | 29 | 30 |
| Ethyl Alcohol | 250.0 | — | — | — | — | — | — |
| Pluronic F127 | 5.0 | — | — | — | — | — | — |
| Sodium | 1.5 | — | — | — | — | — | — |
| benzoate | | | | | | | |
| Kelcogel GC- | 0.47 | — | — | — | — | — | — |
| LA gellan gum | | | | | | | |
| Deionized | 461.6 | — | — | — | — | — | — |
| water | | | | | | | |
| Citric acid | 0.1 | — | — | — | — | — | — |
| Sodium citrate | 0.3 | — | — | — | — | — | — |
| 70% Sorbitol | 250.0 | — | — | — | — | — | — |
| solution | | | | | | | |
| Mouthwash | — | 39.0 | 39.0 | 39.0 | 39.0 | 39.0 | 39.0 |
| Base 5 | | | | | | | |
| Zeothix 177 | — | 1.0 | — | — | — | — | — |
| Zeothix 265 | — | — | 1.0 | — | — | — | — |
| Zeodent 165 | — | — | — | 1.0 | — | — | — |
| Zeodent 113 | — | — | — | — | 1.0 | — | — |
| Silica-Al | — | — | — | — | — | 1.0 | — |

With regard to Table 8, a pre-mix was prepared by adding 250 g 70% sorbitol, 1.5 g sodium benzoate, 0.1 g citric acid and 0.3 g sodium citrate to a 1500 ml beaker containing 461.6 g of deionized water. The solution was stirred until the salts dissolved. Separately, 5.0 g of Pluronic F127 was dissolved in 227.0 g of 95% Ethanol with stirring. After the surfactant was completely dissolved, the two solutions were combined. The resulting solution became cloudy, but cleared up after a few minutes of stirring. 0.47 g of Kelcogel® CG-LA was added and the solution was heated to 75° C. with stirring. Once the temperature reached 70° C., the solution was removed from heat. While the solution was still hot, 39.0 g of the pre-mix solution was added to a 50 ml screw top vial containing 1.0 g of silica and it was then stirred. After approximately 10 minutes of stirring, 0.04 g of sodium chloride was added and the solution was stirred for an additional 2.5 hours.

TABLE 9
Kelcogel ® GC-LA Gellan Gum and
Silica in Store-Bought Mouthwash

| Ingredient in | Mouthwash | Ex. | Ex. | Ex. | Ex. | Ex. | Ex. |
|---|---|---|---|---|---|---|---|
| grams | Base 6 | 31 | 32 | 33 | 34 | 35 | 36 |
| LISTERINE ® | 1000.0 | — | — | — | — | — | — |
| Fresh Burst | | | | | | | |
| Mouthwash | | | | | | | |
| Kelcogel CG- | 2.0 | — | — | — | — | — | — |
| LA gellan gum | | | | | | | |
| Mouthwash | — | 39.0 | 39.0 | 39.0 | 39.0 | 39.0 | 39.0 |
| Base 6 | | | | | | | |
| Zeothix 177 | — | 1.0 | — | — | — | — | — |
| Zeothix 265 | — | — | 1.0 | — | — | — | — |
| Zeodent 165 | — | — | — | 1.0 | — | — | — |
| Zeodent 113 | — | — | — | — | 1.0 | — | — |
| Silica-Al | — | — | — | — | — | 1.0 | — |

With regard to Table 9, a pre-mix was prepared by adding 0.5 g of Kelcogel® CG-LA gellan gum to 1000 g of Fresh Burst Listerine. The solution was stirred for approximately 15 minutes and was then heated with continued stirring to 65°. Once 65° C. was reached, the solution was removed from heat. While the solution was still hot, 39.0 g of the pre-mix solution was added to a 50 ml screw top vial containing 1.0 g of silica and it was then stirred. After approximately 10 minutes of stirring, 0.04 g of sodium chloride was added and the solution was stirred for an additional 2.5 hours. Each of Examples 1-36 exhibited a viscosity of about 2,000 cps at room temperature and standard pressure.

TABLE 10
Keldent ® Xanthan Gum and Silica in Synthesized Mouthwash

| | Mouthwash | Example | Example |
|---|---|---|---|
| Ingredient | Base 7 | 37 | 38 |
| Ethyl Alcohol (g) | 227.0 | — | — |
| Pluronic F127 (g) | 5.0 | — | — |
| Sodium benzoate (g) | 1.5 | — | — |
| Keldent (g) | 2.0 | — | — |
| Deionized water (g) | 461.6 | — | — |
| Citric acid (g) | 0.1 | — | — |
| Sodium citrate (g) | 0.3 | — | — |
| 70% Sorbitol solution (g) | 250.0 | — | — |
| Mouthwash Base 7 (g) | — | 39.0 | 39.0 |
| Calcium silicate 600 (g) | — | 1.0 | — |
| CA Silicate-PA 600 (g) | — | — | 1.0 |

With regard to Table 10, a pre-mix was prepared by adding 2.0 g of Keldent® xanthan gum to a water/alcohol/sorbitol solution containing 461.6 g deionized water, 250 g of 70% sorbitol, 227 g ethyl alcohol, 5.0 g Pluronic F127, 1.5 g sodium benzoate, 0.3 g sodium citrate and 0.1 g citric acid. The solution was stirred in a 1500 ml beaker overnight. To prepare the individual silica samples, 39.0 g of pre-mix was added to a 50 ml screw top vial containing 1.0 g of silica and it was then stirred for approximately 2.5 hours.

### Storage Stability

Each of these Examples was then tested for storage stability over certain time periods. The samples were prepared and left at room temperature for the period of time noted below. After each time period passed, the samples were empirically observed for any separation and/or settling of the particulate silica material from the liquid mouth rinse compositions. The solutions were thus evaluated for their ability to suspend the silica or silicate particles. Rating scales were developed for separation and settling and they are summarized below.

| Rating | Separation-Description | Settling-Description |
|---|---|---|
| 5 | One continuous solution is | No particulate is observed on the |
| | observed. | bottom of the container. |
| 3 | Slight separation is observed | A small quantity of particulate is |
| | near the top of the solution. | observed on the bottom of the |
| | | container. |
| 1 | Noticeable separation is | A large quantity of particulate is |
| | observed in the solution | observed on the bottom of the |
| | | container. |

In the table below, X denotes separation and Y settling.

TABLE 11
Mouthwash Storage Stability

| | 1 day | | 3 day | | 1 week | | 2 week | | 3 week | |
|---|---|---|---|---|---|---|---|---|---|---|
| Ex. No. | X | Y | X | Y | X | Y | X | Y | X | Y |
| 1 | 5 | 5 | 5 | 5 | 5 | 5 | 5 | 5 | 5 | 5 |
| 2 | 5 | 5 | 5 | 5 | 5 | 5 | 5 | 5 | 5 | 5 |
| 3 | 5 | 5 | 5 | 5 | 5 | 5 | 5 | 5 | 5 | 5 |
| 4 | 5 | 5 | 5 | 5 | 5 | 5 | 5 | 5 | 5 | 5 |
| 5 | 5 | 5 | 5 | 5 | 5 | 5 | 5 | 5 | 5 | 5 |
| 6 | 5 | — | 5 | — | 5 | — | 5 | — | 5 | — |
| 7 | 5 | 5 | 5 | 5 | 5 | 5 | 3 | 5 | 1 | 5 |
| 8 | 5 | 5 | 5 | 5 | 5 | 5 | 5 | 5 | 5 | 5 |
| 9 | 5 | 5 | 5 | 5 | 5 | 5 | 5 | 5 | 5 | 5 |
| 10 | 5 | 5 | 5 | 5 | 5 | 5 | 5 | 3 | 5 | 1 |
| 11 | 5 | 5 | 5 | 5 | 5 | 5 | 5 | 5 | 5 | 3 |
| 12 | 5 | — | 5 | — | 5 | — | 5 | — | 5 | — |
| 13 | 5 | 5 | 5 | 5 | 5 | 5 | 5 | 5 | 5 | 5 |
| 14 | 5 | 5 | 5 | 5 | 5 | 5 | 5 | 5 | 5 | 5 |
| 15 | 5 | 5 | 5 | 5 | 5 | 5 | 5 | 5 | 3 | 5 |
| 16 | 5 | 5 | 5 | 5 | 5 | 5 | 5 | 5 | 5 | 5 |
| 17 | 5 | 5 | 5 | 5 | 5 | 5 | 5 | — | 5 | — |
| 18 | 5 | — | 5 | — | 5 | — | | | | |
| 19 | 5 | 5 | — | — | 5 | 5 | 3 | 3 | 3 | 3 |
| 20 | 5 | 5 | — | — | 3 | 3 | 3 | 3 | 3 | 3 |
| 21 | 5 | 5 | — | — | 3 | 1 | 3 | 1 | 3 | 1 |
| 22 | 5 | 5 | — | — | 5 | 1 | 3 | 1 | 3 | 1 |
| 23 | 5 | 5 | — | — | 1 | 1 | 1 | 1 | 1 | 1 |
| 24 | 5 | — | — | — | 5 | — | 5 | — | 5 | — |
| 25 | 5 | 5 | 3 | 5 | 3 | 5 | 3 | 5 | 3 | 5 |
| 26 | 5 | 5 | 5 | 5 | 5 | 5 | 5 | 5 | 5 | 5 |
| 27 | 5 | 5 | 5 | 5 | 5 | 5 | 3 | 5 | 3 | 5 |
| 28 | 5 | 5 | 5 | 5 | 5 | 5 | 5 | 5 | 5 | 5 |
| 29 | 5 | 5 | 3 | 5 | 3 | 5 | 3 | 5 | 3 | 5 |
| 30 | 5 | — | 5 | — | 5 | — | 5 | — | 5 | — |
| 31 | 5 | 5 | — | — | 5 | 5 | 3 | 5 | 3 | 5 |
| 32 | 5 | 5 | — | — | 3 | 5 | 3 | 5 | 3 | 5 |
| 33 | 5 | 5 | — | — | 5 | 5 | 5 | 5 | 5 | 5 |
| 34 | 5 | 5 | — | — | 5 | 5 | 5 | 5 | 5 | 5 |
| 35 | 5 | 5 | — | — | 5 | 5 | 5 | 5 | 5 | 5 |
| 36 | 5 | — | — | — | 5 | — | 5 | — | 5 | — |

Likewise, Examples 37 and 38 exhibited excellent separation and settling properties aver the same time periods.

The vast majority of these compositions showed excellent storage stability with any differences attributable to the apparent difference in gum suspension components. Thus, testing was then undertaken to tooth surface affinity of adduct-treated precipitated silica from mouth rinse compositions as well as the calcium silicate and modified calcium silicate (reacted with phosphoric acid to provide a calcium phosphate/silicate compound for potential tooth remineralization purposes).

### Tooth Surface Affinity

To determine the efficacy of possible deposition of particulates from mouth rinse compositions, specific testing of treated teeth was undertaken. The protocols were as follows:

A previously autoclaved bovine tooth (autoclaved in water, decanted, and stored in methanol) was cut in half lengthwise using a Dremel 400|XPR equipped with a Flex Shaft and a #545 diamond wheel so that the front and back of the tooth surface remained intact. Using the same Dremel setup except equipped with a #8193 aluminum oxide grinding stone, the enamel was ground off the surface of the tooth down to the dentin (visible color change from white to yellow). Once the dentin was exposed, the surface was sanded using progressively finer grits of silicon carbide sandpaper (220 to 400 grit). The dentin was then polished using first a silica flour paste followed by a paste of calcium carbonate (HUBERCAL® 950 from J.M. Huber Corporation), with rinses following each polish. The tooth was placed in a 250-mL beaker and filled with enough 0.5 N HCl to cover the tooth. The tooth was then sonicated for 2 minutes, followed by rinsing with deionized water. The tooth was then allowed to dry. Using the cutting setup above, the tooth was cut in half vertically, followed by the removal of the root. Each side of the tooth (front and back) yielded two usable dentin-exposed pieces.

Teflon tape was cut in half lengthwise and was wrapped around the middle of the polished tooth creating, two exposed and one unexposed sections. The unexposed section was used as a control for comparison during the test. The tooth was gripped along its side with tweezers and was submerged in certain mouthwash examples from above (about 40 mL of each sample mouthwash), that was stirred at approximately 300 RPM with a magnetic stir plate for 60 seconds. During this time, the tooth was moved throughout the subject mouthwash while keeping the dentin portion against the flow. The tooth was then rinsed for two seconds using a squirt bottle of deionized water. After allowing the tooth to dry, the piece was imaged using scanning electron microscopy (SEM). Prior to imaging, the Teflon tape was removed and the exposed portions visually compared to what was covered to assess affinity.

### DETAILED DESCRIPTIONS OF THE DRAWINGS

For each of the provided FIGS. 1-7, the images are arranged as follows: 1) the left side of the image shows the image of the unexposed section of the tooth, 2) the center of the image shows the image of the boundary between the unexposed and exposed sections, and 3) the right side of the image shows the image of the exposed section of the tooth.

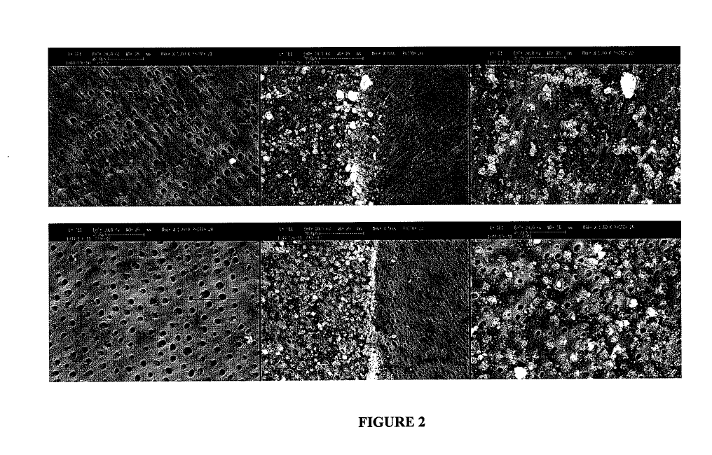

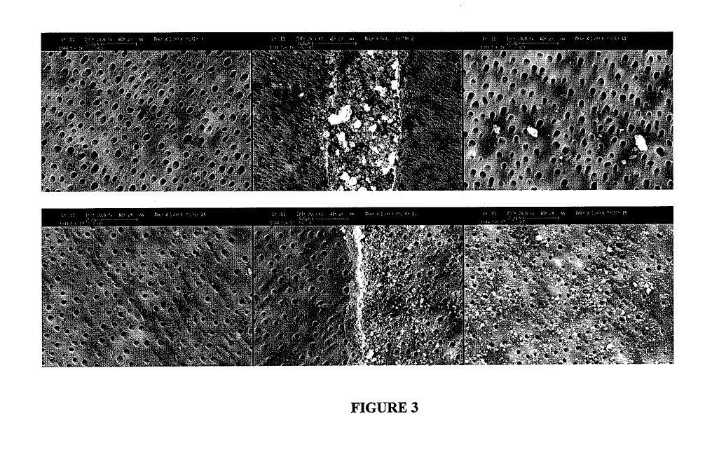

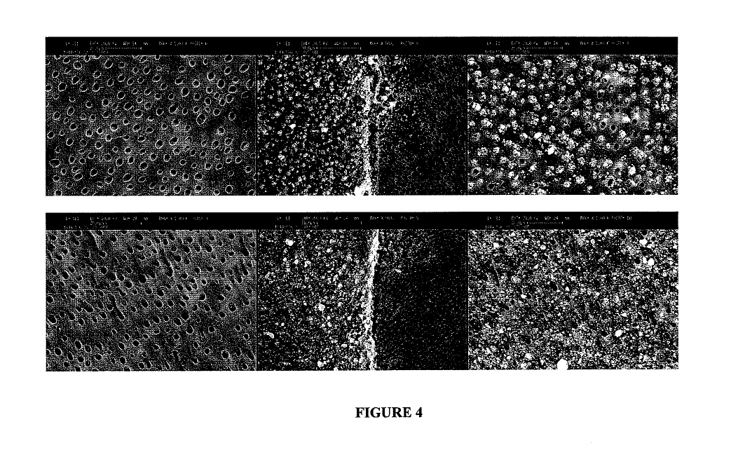

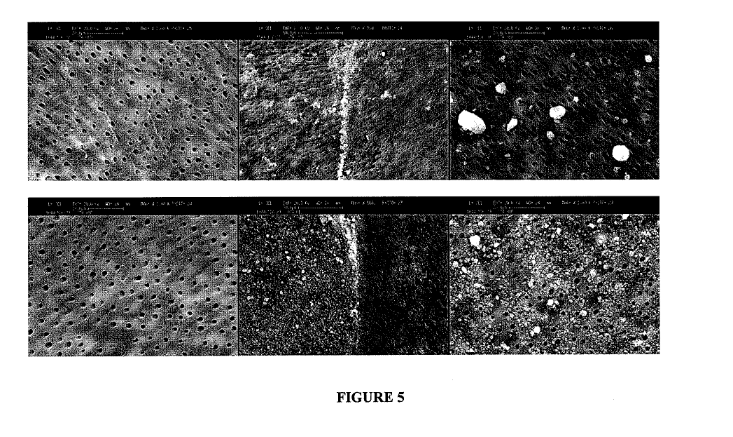

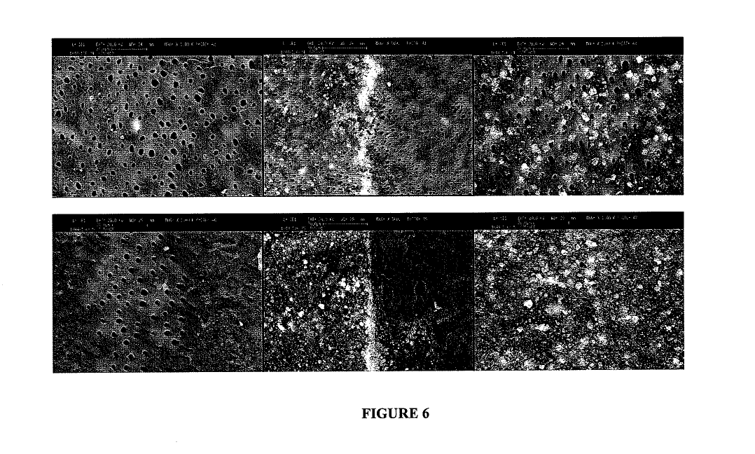

From the images shown in these FIGS. 1-6, it can be seen that the top set of photomicrographs show unmodified silica exhibiting much less affinity than the modified silica (adduct-treated precipitated silica) of the bottom set of photomicrographs (FIG. 1 shows Example 4 on top and Example 5 on the bottom; FIG. 2 shows Example 10 on top and Example 11 on bottom; FIG. 3 shows Example 16 on top and Example 17 on bottom; FIG. 4 shows Example 22 on top and Example 23 on bottom; FIG. 5 shows Example 28 on top and Example 29 on bottom; and FIG. 6 shows Example 34 on top and Example 35 on bottom). The modified silica materials all appear to best adhere to the surface of the tooth while, although a certain degree of adhesion is present for the comparative, non-modified silica particles, there is less propensity, in each instance, for such a solid particulate to enter the dentinal tubules as well as adhere to the exposed tooth surface. As such, it is apparent that the modified silica can actually provide some degree of reliability in settling out of the mouthwash (in suspended form) and adhere to, and potentially provide effective therapeutic benefits from such a mouthwash application and rinsing method to a user's teeth.

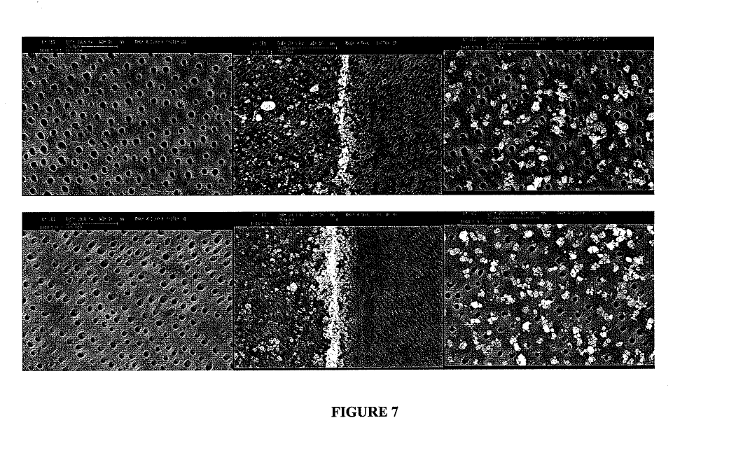

In FIG. 7, the top view is of the calcium silicate product alone and the bottom view of the phosphate-modified calcium silicate material. It appears that the both the silicate materials exhibit an improved affinity for the target tooth surface as particles adhere to the surface as well as enter the tubules in a regular fashion.

Thus, in terms of providing an effective method of delivering solid particles to tooth surfaces through a mouthwash application, the utilization of a proper suspension system to assure uniform distribution of the desired solid particles from a very low viscosity liquid formulation provides the initial results to that end. Additionally, particles, either silica or silicate in nature, accords the necessary affinity for target tooth surfaces (at least) to impart such potentially therapeutic benefits via a solid particulate carrier or by utilization of modified solid particles themselves.

It will be appreciated by those skilled in the art that changes could be made to the embodiments described above without departing from the broad inventive concept thereof. It is understood therefore that this invention is not limited to the particular embodiments disclosed, but it is intended to cover modifications within the spirit and scope of the present invention as defined by the appended claims.

We claim:

1. A mouth rinse composition having a viscosity of at most 10,000 cps at 25° C. and 1 atmosphere pressure and comprising (i) a base solvent and (ii) from 0.1 to 25% by weight of particulate material wherein the particulate material exhibits an affinity for adhesion to tooth surfaces when applied through standard mouth rinsing procedure and the particulate material does not exhibit appreciable separation or settling from within the mouth rinse composition after at least 3 weeks of storage at room temperature.

2. The composition of claim 1 wherein the base solvent is water, alcohol, or a mixture thereof.

3. The composition of claim 1 wherein the particulate material is selected from the group consisting of precipitated silica, silicate, and any mixtures thereof;

4. The composition of claim 3 wherein the precipitated silica comprises an adduct present on at least a portion of the surface of the precipitated silica material to form an adduct-treated precipitated silica material that exhibits a zeta potential reduction of greater than 10% of the zeta potential of a similar precipitated silica material on which no adduct is present.

5. The composition of claim 4 wherein the adduct-treated precipitated silica material exhibits a zeta potential reduction greater than 15% when compared to a precipitated silica material of the same structure on which no adduct is present.

6. The composition of claim 4 wherein the adduct-treated precipitated silica material exhibits a zeta potential reduction greater than 20% when compared to a precipitated silica material of the same structure on which no adduct is present.

7. The composition of claim 4 wherein the adduct-treated precipitated silica material exhibits a zeta potential reduction greater than 25% when compared to a precipitated silica material of the same structure on which no adduct is present.

8. The composition of claim 4 wherein the adduct is a metal element.

9. The composition of claim 8 wherein the metal element is selected from the transition metals or post-transition metals.

10. The composition of claim 9 wherein the metal element is selected from the group consisting of aluminum, zinc, tin, strontium, iron, copper and mixtures.

11. The composition of claim 3 wherein the silicate is calcium silicate or calcium silicate that has been modified by reaction with phosphoric acid.

12. The composition of claim 1 further comprising a suspending aid selected from the group consisting of xanthan gum, high acetyl gellan gum, low acetyl gellan gum, carboxymethylcellulose or mixtures thereof.

13. A method of adhering particulate materials to a tooth surface within a person's oral cavity, wherein the method comprises providing a mouth rinse composition as in claim 1, transferring at least a portion of the mouth rinse composition to the person's oral cavity, maneuvering the mouth rinse composition for a period of time with the person's oral cavity, and expectorating the mouth rinse composition from the person's oral cavity, wherein at least a portion of the particulate materials present within the mouth rinse composition within the person's oral cavity adheres to at least one tooth surface therein.

14. The method of claim 13 wherein the particulate materials impart a therapeutic benefit to the at least one tooth surface.

15. The method of claim 14 wherein the therapeutic benefit is to reduce sensitivity or promote remineralization of the tooth surface.
